(19) United States
(12) Patent Application Publication
(10) Pub. No.: US 2014/0336611 A1
(43) Pub. Date: Nov. 13, 2014

(54) Catheter sheath and tip

(71) Applicant: John R. Lizak, Liitz, PA (US)

(72) Inventor: John R. Lizak, Liitz, PA (US)

(21) Appl. No.: 14/269,104

(22) Filed: May 3, 2014

Related U.S. Application Data
(60) Provisional application No. 61/820,236, filed on May 7, 2013.

(51) Int. Cl. A61M 25/00

(52) U.S. Cl. CPC A61M 25/007
USPC 604/500; 604/523

(57) ABSTRACT

A method and apparatus for diverting the path of an object moving within a catheter sheath so that the object exits the catheter sheath at an angle greater than 0° and less than 90° is disclosed. The subject matter of this application more specifically pertains to catheter sheaths and catheter sheath portions that comprise a lateral opening and an deflection means such that objects moving along the longitudinal axis of a catheter are diverted from the longitudinal axis so that they exit the catheter through a lateral opening in the catheter sheath or catheter sheath portion.

### CLAIM OF PRIORITY

Applicant claims priority to U.S. Provisional Patent Application 61/820,236, entitled “Catheter,” and filed on May 7, 2013.

### FIELD OF THE INVENTION

The subject matter of this application related to snare devices comprising wire cutting loops contained in a flexible lumen which are used for performing endoscopic polypectomy procedures. In particular, the disclosed subject matter pertains to a certain methods and apparatuses that cause a wire cutting loop to be ejected from the flexible lumen at an angle greater than 0° but less than 90°, thereby increasing user efficiency and allowing otherwise difficult to snare polyps to be more easily excised.

### BACKGROUND

Endoscopes are commonly used in medical procedures to allow the operator to look inside a patient through a relatively small incision or natural opening such as the throat or colon.

An endoscope may solely comprise a fiber-optic camera and light source if used for diagnostic purposes, Commonly an endoscope is further associated with an excision means by which an intestinal polyp, or other target tissue, can be removed. Such excision means may cauterize the target tissue, while others comprise wire snares used to surround the tissue and cut it as the snare closes on the tissue.

Endoscopic cauterization is performed by causing the tip of a probe near the target tissue to heat to an appropriate temperature. Although effective, such cauterization can easily cause unintended heat damage to the surrounding tissue.

Other excision means comprise wire snares having a C- or U-shaped loop, or snare, on one end (distal) and a operating means on the other (proximal) end. In operation, the loop is extended and placed over a polyp or other target and then retracted, causing the loop to tighten and cut, or grip, the target. The wire is preferably made from a material, commonly a metal or metal alloy, that has some elastic or shape memory characteristic. The wire is housed within a catheter sheath so that the loop portion is fully within the sheath when retracted. The proximal end of the wire is attached to an operating means that pushes the wire out of the catheter sheath to expose the loop on the distal end and retracts the wire in response to the user's actions. When extended, the longitudinal axis of the wire and loop, is approximately the same as that of the end of the catheter sheath. The use of wire snares avoids the risk of burning the surrounding tissue, but it can be difficult to loop the snare around the target tissue or polyp. Ideally, a polyp's longitudinal axis is perpendicular to that of the catheter sheath so that the wire snare can be extended and easily looped around the polyp for excision. In practice however, polyps are rarely perfectly positioned, especially so when found in the labyrinthine intestinal lumen. When a surgeon encounters a polyp in a difficult to loop orientation, the catheter may need to be rotated on it's longitudinal axis or retracted, at least partially, and reinserted relying on this repositioning to alter the relative positions of the wire loop on the polyp.

### SUMMARY

The subject matter of this application pertains to devices and methods for providing a wire loop cutting tool that is better suited for placement around a colorectal polyp during an endoscopic polypectomy procedure. More particularly, this subject matter of this application concerns novel designs of snare catheters in which the distal end of a wire comprising a U- or C-shaped loop and contained in the catheter's flexible sheath when retracted, can extend from the catheter sheath at an angle greater than 0°, but less than 90° relative to the longitudinal axis of the catheter when operated by the user. In particular, the subject matter of this application pertains to design changes in the distal terminus of a catheter that alter the path of a wire loop exiting a catheter so that the longitudinal axis of the extended loop is at an angle to the catheter.

### BRIEF DESCRIPTION OF THE DRAWINGS:

FIG. 1 illustrates a polypectomy snare extending from the side of a catheter according to the subject matter of this application.

FIG. 2 illustrates the cross section of distal tip of a currently available catheter with a retracted polypectomy snare.

FIG. 3 illustrates the cross section of a distal tip of a currently available catheter with an extended polypectomy snare.

FIG. 4 illustrates the cross section of a catheter comprising an embodiment of the subject matter of this application.

FIG. 5 illustrates the cross section of another embodiment of the subject matter of this application.

### DETAILED DESCRIPTION OF THE PREFERRED EMBODIMENTS

The following description and drawings referenced therein illustrate embodiments of the application's subject matter. They are not intended to limit the scope. Those familiar with the art will recognize that other embodiments of the disclosed method are possible. For example, although reference is made to using the subject matter of this application to excise colorectal polyps, it could also be used for other surgical applications. Further, the subject matter of this application could be used in a wide range of non-surgical applications. All such alternative embodiments should be considered within the scope of the application's claims.

As used in this application: the nouns wire loop, polypectomy snare, snare, cutting loop, and loop may be used to describe the same thing.

Each reference number consists of three digits. The first digit corresponds to the figure number in which that reference number is first shown. Reference numbers are not necessarily discussed in the order of their appearance in the figures.

This application discloses a novel deflection means that changes the trajectory of a snare as it exits a catheter sheath. This application discloses a catheter, in particular, the distal terminal portion of a catheter. The catheter comprises an extendible excision means, such as a polypectomy snare or similar. Unlike other such catheters in which the extendible excision means extends from an opening at the tip of the catheter sheath, the subject matter of this application discloses a catheter terminus comprising an opening lateral to the catheter and a guiding means that causes the extendible excision means to exit the the catheter sheath through the lateral opening.

Catheters generally comprise a flexible tube which may be several meters in length. Catheters have a distal end and a proximal end. The distal end of a surgical catheter is the end that is inserted into a patient. The proximal end remains outside of the patent and is attached to an operating means. Catheters used for wire-loop polypectomy contain a wire preferably of metal or a metal allow that has some degree of structural memory. This wire's distal terminus comprises a loop that is compressed and located within the distal terminal portion of the catheter when the wire is retracted. The wire's proximal end is also attached to said operating means. When the surgeon acts on the operating means to extend the loop the wire is pushed out of its position within the catheter sheath and the loop expands to its un-hindered ovoid shape. Typically, the catheter is associated with, or attached to, a probe further comprising a camera and a light source.

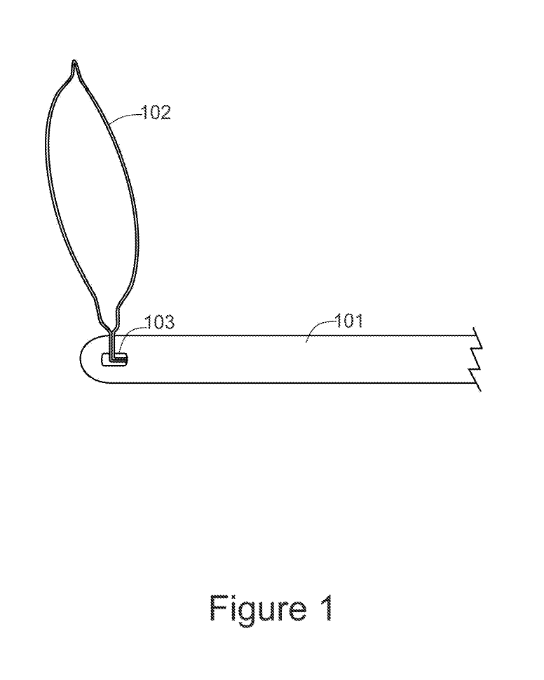

FIG. 1 depicts the terminal portion of a catheter (101) employing an embodiment of the subject matter of this application and showing the extendible excision means (102) extending from the lateral opening (103).

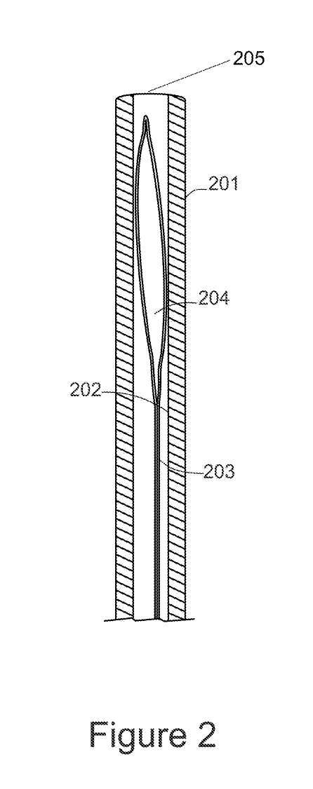

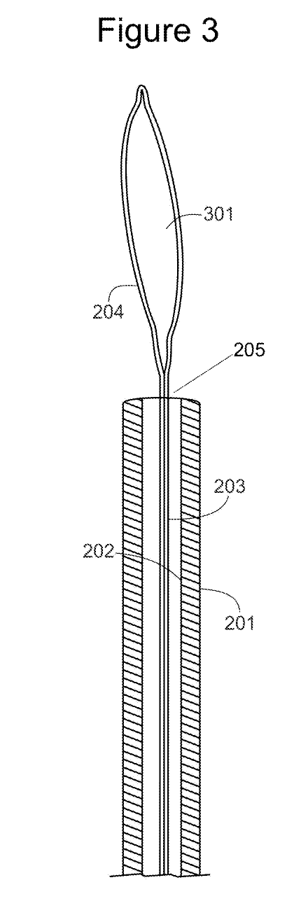

FIGS. 2 and 3 are illustrations of a cross-section of a currently available catheter not practicing the subject matter of this application, and are provided as a reference. The catheter sheath has an external face (201) and an internal face (202), the internal face defining a lumen or channel. The catheter further comprises a wire (203) within the channel and an opening at the distal tip of the sheath of sufficient dimensions to allow the movement of the wire though the opening when extended by the user (205). The distal terminus of the wire comprises an excision means, here a polypectomy snare (204), that is constrained by the internal face of the catheter sheath when the wire is retracted. The catheter is normally flexible to allow it to readily bend as necessary to be guided by the user.

Both currently available catheters and catheters practicing the subject matter of this application comprise a longitudinal axis defined by the distal portion of the catheter. In currently available catheters, when the user extends the wire, the snare moves out of the opening at the distal tip of the sheath in line with the longitudinal axis and expands, forming a snare (301) which may be looped around a polyp or other target and retracted. Retraction of a snare may cut the targeted tissue, or may grasp the targeted object for removal such as during an endoscopic removal of foreign bodies. The catheters further comprises a transverse axis perpendicular to the catheter's longitudinal axis. The catheter sheath and it's lumen are substantially circular.

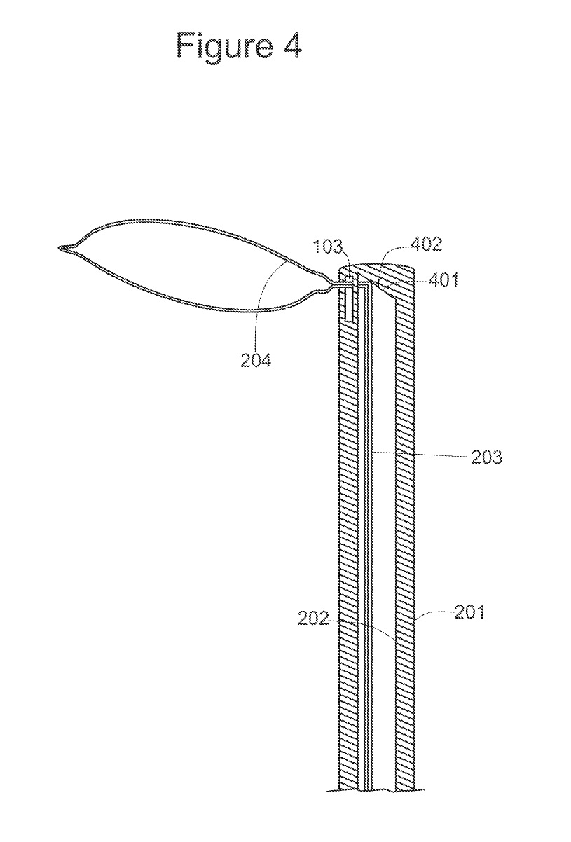

The subject matter of this application differs from other catheters in that when the extendible excision means is extended, it is guided by a guiding means (401) so that the extendible excision means exits the catheter's sheath through a lateral opening (103). In the embodiment depicted in FIG. 4, the guiding means and lateral opening is formed in situ at the distal end of the catheter. In a most preferred embodiment, this lateral opening is located near (meaning close to, or at) the most distal point of the catheter, is annular and has a diameter substantially equal to the inner diameter of the catheter. The opening at the distal terminus located along the catheter sheath's longitudinal axis commonly found in currently available catheters is absent or closed. In the embodiment illustrated by FIG. 4, the guiding means is an angled plane (402). In preferred embodiments, the angle of this plane is approximately 45° so that when the loop is extended, it contacts the angled plane and is deflected from its path in line with the longitudinal axis of the catheter to a lateral path and exits the catheter sheathsheath through the opening in the catheter sheath (103). Other embodiments may comprise guiding means having planes of other angles, thereby causing the extendible excision means to exit the lateral opening at different angles respective to the longitudinal axis of the catheter sheath, however, in all useful embodiments, the guiding means and the lateral opening must align so that the excision means is deflected through the lateral opening when extended.

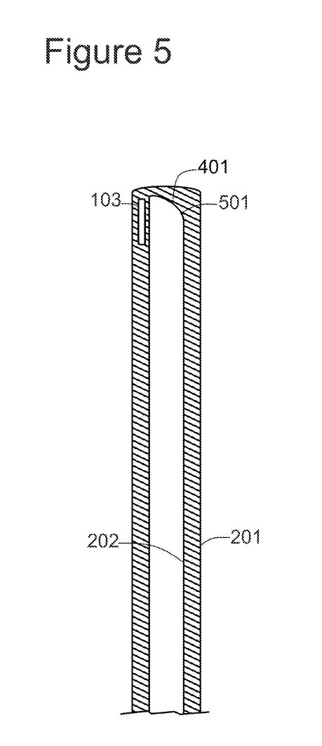

Alternative embodiments are depicted in FIG. 5. In these embodiments, a distal catheter tip is manufactured or formed comprising a guiding means and lateral opening as disclosed. This distal tip may be attached to the distal end of a currently available catheter by fixation means known in the relevant arts, thereby becoming the new distal terminus of the catheter. In most preferred designs of this separately manufactured embodiment, the inner diameter of the distal tip is the same, or slightly larger than the outer diameter of the catheter so that the distal end of the catheter may be placed inside the catheter tip.

In another preferred embodiment, the guiding means comprises an annular segment (501) that is aligned with the lateral opening (103) so that when the loop is extended, it contacts the annular segment and is deflected from its path in line with the longitudinal axis of the catheter to a lateral path and exits the catheter sheath through the opening in the catheter sheath (103). The angled plane and annular segment guiding means may be used with either the in situ or distal tip embodiments.

I claim:

1. A catheter sheath comprising a longitudinal axis and a transverse axis, a distal terminus and a proximal terminus, said sheath further comprising an outer surface and an inner surface, said inner surface defining an inner annular lumen; said sheath further comprising
a. a closed distal terminus,
b. a lateral annular opening spanning the sheath's inner surface and outer surface, said annular opening being located near the closed distal terminus and having a diameter substantially the same as the catheter sheath's said inner annular lumen, and
c. A deflection means contained within said catheter sheath and located near the closed distal terminus such that an object moving towards said sheath's distal terminus along said longitudinal axis is deflected by the deflection means such that said object exits the catheter sheath though said lateral annular opening.

2. The catheter sheath of claim 1 in which the said deflection means is an angled plane.

3. The catheter sheath of claim 1 in which the said deflection means in an annular segment.

4. A catheter sheath portion comprising a longitudinal axis and a transverse axis, a distal terminus and a proximal terminus, said sheath portion further comprising an outer surface and an inner surface, said inner surface defining an inner annular lumen; said sheath portion further comprising
a. a closed distal terminus,
b. a lateral annular opening spanning the sheath portion's inner surface and outer surface, said annular opening being located near the closed distal terminus and having a diameter substantially the same as the catheter sheath portion's said inner annular lumen, and
c. A deflection means contained within said catheter sheath portion and located near the closed distal terminus such that an object moving towards said sheath portion's distal terminus along said longitudinal axis is deflected by the deflection means such that said object exits the catheter sheath portion though said lateral annular opening.

5. The catheter sheath portion of claim 1 in which the said deflection means is an angled plane.

6. The catheter sheath portion of claim 1 in which the said deflection means in an annular segment.

7. A method of diverting the path of an object traveling along the longitudinal axis of a catheter sheath comprising the steps of:
a. affixing the catheter sheath portion of claim 4 to the distal terminus of a catheter.
